(19) United States
(12) Patent Application Publication
(10) Pub. No.: US 2005/0048089 A1
(43) Pub. Date: Mar. 3, 2005

(54) Topical delivery system containing colloidal crystalline arrays

(76) Inventors: Daniela Bratescu, Northport, NY (US); George Cioca, Lake Grove, NY (US); Lilliana George, Centerport, NY (US); Vasile Ionita-Manzatu, Old Bethpage, NY (US); Charles Craig Tadlock, Islip Terrace, NY (US); Heather Bowen-Leaver, South Huntington, NY (US)

Correspondence Address: THE ESTEE LAUDER COS, INC, 125 PINELAWN ROAD, MELVILLE, NY, 11747, US

(21) Appl. No.: 10/922,464

(22) Filed: Aug. 20, 2004

Related U.S. Application Data
(60) Provisional application No. 60/497,257, filed on Aug. 22, 2003.

(51) Int. Cl. A61K 6/00; A61K 7/00

(52) U.S. Cl. 424/401

(57) ABSTRACT

The present invention relates to a topical system for application to the skin comprising an effective amount of a colloidal crystalline array in a hydrophilic phase, and at least one oil-containing phase.

### FIELD OF THE INVENTION

The invention relates to compositions for topical application to the skin. More specifically, the invention relates to topical compositions containing colloidal crystalline arrays.

### BACKGROUND OF THE INVENTION

Colloidal crystalline arrays (CCA) are composed of monodisperse, charged spherical particles that, under proper ambient conditions, self-assemble into a crystalline lattice having unusual properties, particularly in the production of color. The color of the CCA coloring system is produced as light travels through and is diffracted by the crystalline structure of the CCA. The uniform particle size and surface charge density of the spheres cause coulombic electrostatic repulsive forces between them and allow the spheres to “self-assemble” into crystalline lattice structures which efficiently diffract light meeting the Bragg condition. See Asher, S. A., et al, “Novel Optically Responsive and Diffracting Materials Derived from Crystalline Colloidal Array Self-Assembly”, Chapter 33, ACS Symposium Ser., pps. 495-506 (1997); incorporated herein by reference. Bragg's law is represented by the equation, mλ₀=2nd sin θ; where, m is an integer representing the number of planar layers of the CCA, λ₀ is the wavelength of light in a vacuum, n is the refractive index of the system, d is the interplane spacing, and θ is the Bragg angle. Bragg diffraction of light occurs from planes of closely-packed spheres in succession and in parallel alignment to a surface. See Tse, Albert S., Wu, Zhijun, and Asher, Sanford A., “Synthesis of Dyed Monodisperse Poly(methyl methacrylate) Colloids for the Preparation of Submicron Periodic Light-Absorbing Arrays”, Macromolecules 28, pps. 6533-6538 (1995); incorporated herein by reference.

The spheres arrange themselves in an order such that there are at least two planes running through the array. Each of the planes is parallel to one another and has an angle incident thereto. The distance between the planes is determined by the number density of the particles, the particle size and the surface charge. Because the spacing of CCAs is similar to the wavelength of visible and near-IR light, strong Bragg diffraction of light occurs as it travels through the CCAs. The creation of color, by the self-assembly of the spheres into CCAs, is partially dependent on the concentration density of the spheres.

The order in which the spheres of a CCA arrange themselves is based on the repulsive forces between them. The spheres have a highly uniform surface charge density. They strongly electrostatically repel each other when the space between them is within a Debye layer length (<1 μm). The surface charge density is an estimation of the ionized H⁺ or OH⁻ counter-ions. This estimation can be made using potentiometric or conductometric titration methods known in the art.

The surface charge density is quantified by the equation, σ₀=Nₛev; where, σ₀ is the surface charge density, Nₛ is the number of charged sites per unit area, v is their valency, and e is the fundamental charge on the electron (1.6×10⁻¹⁹ coulomb). The H⁺ or OH⁻ ions are predominantly found on the surface of the sphere on what is commonly referred to as the electrical double layer. The electrical double layer affects the repulsive forces between the spheres and thus, affects their process of self-assembly.

The counter-ion cloud of each sphere surrounds the electrical double layer at the surface of the sphere. When spheres are in close proximity to one another, there is a slight overlap of the counter-ion clouds associated with each of the spheres. Immediately, the spheres repel each other due to the repulsive forces caused by the counter-ions. Scientific Methods for the Study of Polymer Colloids and Their Applications, supra, at 132. The CCA formed by the self-assembly of the spheres is a result of the repulsive forces between them. When the energy is greater than about kT, where k is the Boltzmann constant and T is the absolute temperature, the spheres are able to self-assemble, thereby producing the orderly array that diffracts light to produce an iridescent color.

The unique properties of the CCAs have found a number of practical applications. For example, the synthesis of monodisperse spherical particles and CCAs composed thereof which produce an iridescent color is known and described in, for example, U.S. Pat. Nos. 4,627,689, 4,632,517, and 5,452,123, the contents of which are incorporated herein by reference. In these patents, a crystalline colloidal narrow band radiation filter and methods for making switching devices and related devices using CCAs are disclosed. CCA's have also been disclosed as being useful in cosmetic and pharmaceutical compositions to confer non-pigment based iridescent color to the compositions; see WO 0047167, the contents of which are incorporated herein by reference. A commercially available product called “La Neige” also apparently utilizes CCAs as part of a pore-minimizing cosmetic composition with a hydroalcoholic base.

Although there have been some practical applications of these interesting crystals, their widespread use has been somewhat limited by the delicacy of the system. It has generally been recognized that a substantial lack of ionic impurities in the medium containing the CCAs is necessary to prevent interference with the repulsive forces which are required to maintain the self-assembled lattice structure. In addition, the spheres are traditionally utilized in a substantially hydrophilic, i.e., hydroalcoholic or aqueous, medium, in the absence of any oil or other hydrophobic solvents. These features severely hamper the use of the arrays in topical compositions, because in the absence of oil, the resulting products possess little or no emolliency, leaving an unpleasant end feel on the skin. Use of high levels of alcohols in such products can also be very drying to the skin. In addition, without the presence of oil, the topical product is limited to the use of water or alcohol soluble components, thereby preventing the use of fragrance or essential oils, or oil soluble active components. Thus, CCAs have not to date achieved the broad level of utilization in topical, particularly cosmetic, products that might be expected of a material that has such a uniquely aesthetic appearance. The present invention now provides a novel approach to compositions containing CCAs, as well as unique methods of utilizing such compositions for topical delivery.

### SUMMARY OF THE INVENTION

The present invention relates to a topical system for application to the skin comprising an effective amount of a colloidal crystalline array in a hydrophilic phase, and at least one oil-containing phase. The unique system is useful in a variety of topical applications, which permits unique exploitation of the aesthetic potential of the compositions combined with the greater cosmetic or skin treatment benefits available with an oil-containing system. Among other uses, the system provides a nano-delivery system which permits penetration of actives (both oil- and water-soluble) into the stratum corneum, as well as skin-color correction and unique types of fragrance products.

### DETAILED DESCRIPTION OF THE INVENTION

A. The spheres and their process for assembly

The spheres that are useful in the present invention can be any spheres capable for forming colloidal crystalline arrays. As noted above, the creation of CCAs is well documented, and they can be prepared in accordance with any method of manufacture known in the art. The spheres can be natural or treated cross linked materials or other materials having a refractive index value of greater than about 1.0, preferably between 1.5 and 3.0. The spheres of the CCAs are formed by treating at least one precursor and a surfactant. The general process involves emulsion polymerization or condensation of the precursor and the surfactant to form spherical particles of monodisperse uniform particle size and uniform surface charge density. Known polymerization techniques such as, for example, dispersion or emulsion polymerization or condensation processes are described in Bhattacharyya, Bhupati and Halpern, B. David, “Application of Monodisperse Functional and Fluorescent Latex Particles”, Polymer News 4, pps. 107-114 (1977); incorporated herein by reference. Preparation of CCAs is also described, in U.S. Pat. No. 4,632,517.

Specifically, the spherical particles of CCAs can be formed by combining the precursor and the surfactant with deionized, doubly distilled water and allowing it to polymerize in a water bath until crystal formation is complete, usually about 4 to 8 hours. Crystal formation is verified by the appearance of an iridescent color. The amount and type of precursor, and the amount of surfactant are factors which determine the concentration density of the spheres and consequently, the self-assembly of the spheres into CCAs.

In principle, any one or more organic or inorganic precursors which are capable of combining to form spherical colloidal particles that have a monodisperse uniform particle size and uniform surface charge density can be used in the present invention. The term “monodisperse” as used herein describes a particle size distribution of the spheres which is gaussian and has a low standard deviation (i.e., standard deviation of less than 5 percent of the mean). The precursor can be any material capable of assembling into an ordered array dispersed throughout a solvent.

The precursors can be selected from, for example, methacrylic acid and derivatives thereof such as, for example, polymethylmethacrylate (hereinafter referred to as “PMMA”), silicon oxides and hydroxides such as, for example, silica (e.g. silicon dioxide), aluminum oxides such as, for example, aluminum dioxide, polytetrafluoroethylene, acrylamides, styrene and polymers thereof such as for example, polystyrene, titanium alkoxides such as for example, titania, and divinylbenzene. Such starting materials are disclosed, for example, in U.S. Pat. No. 5,452,123. More preferably, however, the precursor is silica.

The precursor is combined with the surfactant, the amount of which can vary depending on the desired particle size of the spheres. In general, there is an inverse relationship between the amount of surfactant and the size of the spheres (i.e., lower amounts of surfactant produce larger sized spherical particles.) Preferably, the amount of surfactant is about 0.01 to about 10 percent of the weight of the composition. The surfactant has an HLB of greater than about 12. Examples of suitable surfactants include but are not limited to MA-80 which is sodium di(1,3-dimethylbutyl) sulfosuccinate in isopropanol and water, sodium dodecylsulfate, nonoxynol series, octoxynol series, and other surfactants which can be found for example in the CTFA International Dictionary of Cosmetic Ingredients.

The spheres used in the present invention may have an average particle size of about from about 50 to about 1500 nm in diameter. The variation in particle size should preferably be less than about 5 percent of the mean. The uniform particle size promotes the equalization of the repulsive forces between the spheres and therefore, assists the spheres in the process of self-assembly.

Particularly preferred for use in the present invention are silica spheres. Silica spheres of a relatively small size, are particularly preferred. Excellent results occur with silica spheres having an average diameter of from about 50 to about 90 nanometers, more preferably about 60 to about 80 nanometers. Such spheres may be purchased commercially. A particularly useful product is manufactured by C.C.I.C., Osaka, Japan, and is available commercially from Ried International (Brentwood, N.Y.) under the name Fire Crystal 615. This product is a suspension of about 15-17% by weight silicone oxide particles in about 80-85% water, with or without included ion exchange resin. The average particle size diameter is between about 60 nanometers to about 80 nanometers. Such particles yield a very attractive transparent or translucent CCA suspension.

By “effective amount” of particles is meant that amount of particles which confer a color to the composition without the presence of pigment. The effective amount of particles to be used is expressed herein in terms of the total weight of the hydrophilic phase component of the system. The amount of spherical particles used in the compositions of the invention can be between about 0.5 to about 20% by weight of the hydrophilic phase, more preferably 0.5 to about 6%. However, specific ranges within these broad ranges can be used to achieve specific aesthetic and/or visual effects. These specifics of these ranges will be discussed in more detail below for the specific applications desired. The particles are suspended in a hydrophilic phase, which may be aqueous, alcoholic or hydroalcholic, e.g., water, monohydric or polyhydric alcohols, glycerine, or combinations thereof. This hydrophilic phase as a whole typically comprises from about 20 to about 95% by weight of the total system, preferably about 30 to about 90%, more preferably about 40 to about 85%.

B. Oil Phase Components

A unique aspect of the present system is the presence of an oil phase in combination with the hydrophilic phase containing the CCAs. It has been unexpectedly discovered that it is possible to combine substantial quantities of oil with the CCA-containing phase without disrupting the sensitive balance of the self-assembled lattice. The oil phase may comprise one or more of any typically used cosmetically or pharmaceutically acceptable oils, an oil being defined for the present purpose as any pharmaceutically or cosmetically acceptable material which is substantially insoluble in water. A comprehensive listing of oils useful for topical use can be found in International Cosmetic Ingredient Dictionary and Handbook, Ninth Edition, the contents of which are incorporated herein by reference. As oils can perform different functions in the composition, the specific choice is dependent on the purpose for which it is intended. The oils may be volatile or non-volatile, or a mixture of both. For example, suitable oils include, but are not limited to, silicone oils, both cyclic and linear, such as methicone, cyclomethicone, dimethicone (of various viscosities) or phenyl trimethicone; hydrocarbons, such as decane, dodecane, tridecane, tetradecane, isoparaffins, squalane, mineral oil, polyisobutene isotetracosane, or isoeicosane; vegetable oils, such as coconut oil, jojoba oil, sunflower oil, palm oil, olive oil, apricot oil, soybean oil; carboxylic acid esters such as isostearyl neopentanoate, cetyl octanoate, cetyl ricinoleate, octyl palmitate, dioctyl malate, coco-dicaprylate/caprate, decyl isostearate, myristyl myristate; animal oils such as lanolin and lanolin derivatives, tallow, mink oil or cholesterol; glyceryl esters, such as glyceryl stearate, glyceryl dioleate, glyceryl distearate, glyceryl linoleate, glyceryl myristate, and fragrance or essential oils, such as lavender or rose oils.

Also encompassed within the meaning of “oil” in the present invention are polyfluorinated solvents. Examples of useful solvents of this type include, but are not limited to, cosmetically or pharmaceutically acceptable hydrofluoroethers (HFEs, commercially available from 3M); perfluorocycloalkanes (FLUTEC™ products, commercially available from F2 Chemical); perfluoromorpholines such as 4-trifluoromethylperfluoromorpholine and 4-pentafluoroethylperfluoromorpholine; and perfluoroalkanes such as dodecafluoropentane and tetradecafluorohexane.

The system may contain a single oil, but more typically will contain a combination of two or more oils. Although there is no absolute minimum amount of oil that can be used, the system will normally comprise at least about 1% by weight of oil, preferably at least about 3%, more preferably at least about 5%. An excessive amount of oil can potentially disrupt the CCA network, so it is preferred that the amount of oil used does not exceed 80% by weight. The amount and the choice of oils used can affect the visual appearance of the final product, as will be explained in more detail below. The term “system” is used here to describe a variety of different possible physical combinations of the oil and hydrophilic phases. In certain embodiments, and as will be discussed in more detail below, the oil and hydrophilic phases are physically combined in such a way as to appear to the eye as a single phase, much as an emulsion does (although the system is not an emulsion, and does not require the presence of an emulsifier). In another embodiment, the two or more phases are visually separate but in physical contact, and are shaken together just before the application to the skin. In yet another embodiment, the hydrophilic and oil phases are physically separated until the time of application, and then are combined together by simultaneous dispensing, or on the skin, by simultaneous or near simultaneous application of the phases.

C. Additional Components in Oil Phase

As discussed briefly above, the system of the present invention can present a variety of visual effects. In certain embodiments, the system appears as a single phase, with no clear demarcation between oil and hydrophilic phases being visible to the naked eye. In those uses of the system in which such an appearance is important, inclusion of at least about 0.5% to about 5% of a silicone elastomer in the oil phase is helpful in maintaining the stability of the incorporation of the oil phase into the hydrophilic phase. Other nonionic thickeners may also be used, for example nonionic celluloses or polysaccharides. Preferably, thickeners are added to the oil phase.

One of the great advantages of the present system is that it permits the incorporation of a wider variety of active components into a CCA system than has previously been possible. The hydrophilic phase can accept nonionic water soluble actives in an amount of up to about 10%. Useful actives that can be incorporated into the hydrophilic phase are reseveratrol, white birch, centella asiatica, trehalose, sucrose, yeast extract, particularly white wine yeast extract, Particularly preferred actives for inclusion in the hydrophilic phase are bioconverted or fermented materials, such as are described in copending U.S. Ser. No. 10/427,568, the contents of which are incorporated herein by reference.

The presence of an oil phase also permits the addition of oil soluble actives into the system. Any oil soluble actives can be incorporated into the oil phase, in an amount of up to about 5%, preferably from about 1-2% if the product is a one-phase product Examples of useful actives to be delivered by such a system are Vitamin E and oil soluble botanical extracts or derivatives, e.g., Chamomille extract, apricot oil, nut oil, eucalyptus, rosemary oil, argan oil, passion fruit oil, and the like. The person of ordinary skill in the art will readily recognize that the examples presented are non-exhaustive, and that, within the guidelines given, any water soluble or oil soluble active for topical delivery can be employed in the claimed system, including both cosmetic and pharmaceutical actives. Similarly, other cosmetic additives, such as emollients, can also be incorporated into the composition, provided that the additive is sufficiently nonionic to avoid disruption of the CCAs.

The characteristics of the CCAs, and the present system provide a number of heretofore unrecognized applications. It has previously been recognized that the concentration of the spheres in a product can influence the color of the product as a whole and provide an aesthetic visual effect. However, it has now been unexpectedly discovered that the CCAs constitute an effective filter of visible light. In particular, when white light is passed through a CCA-containing composition, light is diffracted in such a way that, depending on the concentration of the spheres, a single color light, different from the visible color of the composition, will shine through. For example, an aqueous composition or the water phase of an oil-containing composition containing from about 0.75% to about 2.6% by weight of silica spheres of a 60-80 nm diameter yields a product that is pink in color, but which permits only an orange-colored light to shine through. A similar composition containing from about 2.7% to about 3.9% of spheres in an aqueous phase produces a green product, but which allows only a red light to shine through it. A composition containing from about 4 to about 6% of spheres yields a blue-purple colored product, which in turn permits only a gold color to pass through. Aside from being a lovely aesthetic effect, this also has an unexpected but very practical application, namely, that the compositions can be used as color correctors on the skin, permitting adjustment of an undesired color on the user's skin. For example, the blue product, which permits golden yellow light to shine through, is an effective corrector of darker, sallow olive skins. The green product, which filters through a red light, provides an very effective corrector of ruddy or reddish skins. Similarly, the pink product provides a balancing correction to very light, pale skins that have little natural color by transmitting an orange light. This effect is observed not only with the oil-containing system, but also with the CCAs alone, using the amount of spheres indicated. Thus, the systems of the invention can be used, in the colors indicated, as a non-pigment containing concealer or color-balancing product that is natural looking, lacking the opacity that can be present in the more traditional concealers or color correctors.

The system of the invention can also be used as a pore minimizer. As noted above, this use for a strictly hydroalcoholic CCA composition is known. However, the present system provides considerable improvement over the known product. The visual pore minimizing is provided by the light diffusing silica particles; however, the present system, in the provision of an oil phase, allows a smoother application, and less gritty feel to the product.

The CCA system can also provide a convenient and unique means of fragrance delivery. The ability to combine an oil phase with the CCAs permits the addition of fragrance components, which are largely oil based. In addition, the presence, in preferred embodiments of volatile silicones and fluorocarbons gives a cool, refreshing feel to the skin upon evaporation of these materials.

The systems of the invention also have the potential for use as a unique delivery system for actives. There is a current trend toward the development of nano-sized delivery systems. Particles in the nano-size range are generally recognized as having a greater potential for tissue penetration than larger particles, and thus may result in a more efficiently targeted delivery of active or other materials. The present system, with its nano-sized particles, can provide the basis for such a delivery system. Active materials can be incorporated onto or into the particles, or within the network provided by the array. The uniformity in size of the spheres also aids in uniform distribution of the particles and thus further enhances the efficacy of active delivery within the target tissue. Thus, the system may also provide a more effective means for delivery of biologically active material to and within the skin, including both pharmaceutical and cosmetic actives.

The system of the invention can be presented in a number of different forms and packaging options. As alluded to previously, it is possible to prepare a homogeneous appearing, “single phase” composition in which there is no visual distinction between the hydrophilic and oil phases. This product will ordinarily be prepared with a relatively low level of oil phase, for example, less than about 20% total of the system will constitute the oil phase, including oils and oil soluble materials in the composition. The “single phase” product will typically contain a least three oils, a polyfluorinated solvent, such as a hydrofluoroether, a second oil selected from among any of the other types of oils, and cyclomethicone or isododecane. The cyclomethicone or isododecane acts effectively as a cosolubilizer to hold the oil phase together and to hold the oil phase together with the hydrophilic phase The polyfluorinated oil(s) will usually be present in an amount of from about 0.5 to about 10, preferably about 1 to about 5%, other oils from about 0.5 to about 10%, preferably from about 1 to about 5%, and the cosolublizer also present in an amount from about 0.5 to 10%, preferably about 1 to 5%. As a general rule, the more viscous the oils being used, the more cosolubilizer that will need to be used. As noted above, in this embodiment, it is preferred to include a silicone elastomer, in an amount of from about 0.5 to 5% in order to further stabilize the system.

When 20% or more of the oil phase is desired, there will be a distinct separation of phases, even if a cosolubilizer is present. Therefore, as an alternative, the system will be presented as a multi-phase product, i.e., one in which the hydrophilic and oil phases appear visually distinct. Within the multi-phase system, there is also a variation in the presentation. For example, the system may contain two visually distinct phases. In the two phase system, one or more oils of any type can be used, provided that the oils are soluble in each other, without the presence of a cosolubilizer, Two variations of this embodiment can be made: one, in which the oil phase appears on top of the hydrophilic phase; in this case the oil phase preferably contains lower viscosity (i.e., <350 cs) silicones, vegetable oils, hydrocarbons, and/or esters. The second variation is one in which the oil phase appears on the bottom; in this case, the oil phase will contain at least one polyfluorinated solvent, or a high viscosity (i.e., ≧350 cs) silicone, which may be combined with any oil(s), such as a silicone or hydrocarbon, that will readily solubilize in them. In such a case, the oil phase may be up to 80% of the product as a whole, but typically, the oil phase will constitute from about 40 to about 60% by weight, and more typically each phase will be present in approximately equal proportions. Another variation is one in which there are three visually distinct phases, In this embodiment, the three phases will comprise the hydrophilic phase, and two oil phases, each one being non-soluble in the other. This arrangement is achieved, for example, by the preparation of one phase containing from about 10 to about 35% of a polyfluorinated solvent and oils soluble therein or a high viscosity silicone, a second oil phase containing from about 10 to about 60% other oils not soluble in the polyfluorinated solvent or silicone, and the hydrophilic phase from about 10 to about 70%. Different visual effects can be achieved here as well. When the polyfluorinated solvent is one phase, this will appear at the bottom, with the hydrophilic phase in the middle and the other oils on top. When the high viscosity silicone is used, the hydrophilic layer is on the bottom, the silicone layer in the middle and the other oils on the top. In all multiple phase embodiments, the phases can be packaged together in a single container, in physical contact and then shaken together just prior to use. They will settle back to their original positions upon resting, and will filter light in the same manner as a single phase-appearing composition. The compositional and application variations available with the present invention provide a variety of different effects, e.g., fragrancing or moisturizing, which heretofore have not been possible with other CCA compositions because of the limitations on the use of oil in the compositions. With the present system, however, it is now possible to include fairly high concentrations of fragrance and/or oil-soluble actives or emollients in the composition.

An alternate option is to provide the individual phases in separate containers or dispensers, so that they are physically separated until just prior to or at the time of application to the skin. Such separation can be accomplished by partitioning of a single container, or by provision of two separate containers, each containing one of the phases. Application of the system in this case will employ, for example, a pump dispensing simultaneously from the partitioned chambers, or a simultaneous or nearly simultaneous dispensing from separate containers onto the skin, and mixing of the phases thereon. This arrangement is most useful when a high level of oil in the system is desired, or when the oil phase is part of an emulsion, e.g., a nanoemulsion, which will contain surfactants or emulsifiers that may be disruptive to the CCA structure.

A complication of the use of the CCAs in commercial application is their sensitivity to impurities in the medium in which they are prepared. Stability of the CCAs requires a highly pure medium with a low ionic strength due to a low level of ionic impurities. If the ionic strength is too high, flocculation may occur and the color dissipates. Preferably, the medium has a conductometric reading of less than about 2.5 μΩ⁻¹ indicating that the ionic purity of the medium is sufficient for CCAs to form. More preferably, the medium is non-ionic. In practice, it may be difficult to keep the medium of a packaged commercial product so pristine for prolonged periods of storage. It is therefore preferred, in cases where the maintenance of a non-ionic environment is not certain, that the package containing the CCA system of the invention be supplied with an ion exchange resin equipped to pull ions from the medium so as to maintain the stability of the CCA.

The ion exchange resin utilized should be a mixed resin containing both cationic and anionic exchange capabilities, so that ions of all types are removed from the medium. Preferably, the resin has a 1:1 stoichiometric ratio, i.e., 1 equivalence of cation equilibrium capacity to 1 equivalence of anion equilibrium capacity. Such resins are readily available commercially, for example, Diaion® SMNUP from Mitsubishi Chemical, or AG 501-X8 and Bio-Rex MSZ 501(D) from Bio-Rad. The resin must be in substantial contact with the fluid medium to be effective. The resin can be included directly into the product, although aesthetically, this is less desirable. Thus, in order to retain the most attractive appearance of the product, it is preferred that the resin remain separate but in contact with the product. Thus, a screened or porous region of the package may contain the resin, so as to permit access of the fluid to the resin, but to prevent the particles of the resin from entering the product. An alternate form of contact may be provided by inclusion of a separate porous bag or similar enclosure for the resin, which enclosure has sufficient strength to contain the resin in the aqueous environment, but which is sufficiently “open” to permit continuous access of the CCA fluid to the enclosed resin. The resin is typically used in an amount of at least about 1%, and preferably at least about 3 to 4%, but higher amounts can also be used.

The invention is further illustrated by the following non-limiting examples:

### Example 1

This example illustrates compositions of the invention in different colors:

| | Material | Weight % |
|---|---|---|
| A. Green | Fire Crystal 615 | 17.00 |
| | (water 85%/15% silicon dioxide) | |
| | Denatured alcohol | 8.50 |
| | Purified water | 59.50 |
| | Methyl Perfluorobutyl ether | 6.70 |
| | Cyclomethicone/polysilicone-11 | 2.25 |
| | Cyclomethicone | 3.00 |
| | Dimethicone, 5 cs | 3.00 |
| B. Pink | Fire Crystal 615 | 13.28 |
| | Denatured alcohol | 8.30 |
| | Purified water | 61.42 |
| | Methyl perfluorobutyl ether | 10.00 |
| | Cyclomethicone | 4.50 |
| | Dimethicone, 5 cs | 2.50 |
| C. Blue(fragranced) | Fire Crystal 615 | 12.60 |
| | Denatured alcohol | 8.50 |
| | Purified water | 62.90 |
| | Methyl perfluorobutyl ether | 6.75 |
| | Cyclomethicone/Polysilicone-11 | 2.25 |
| | Cyclomethicone | 3.00 |
| | Dimethicone, 5 cs | 3.00 |
| | Fragrance | 1.00 |

The general procedure for preparation of the compositions is as follows:
1. In a main kettle, water phase ingredients are added, in combination with 4% by weight ion exchange resin.
2. Mixing is started under moderate agitation, and the mixture is warmed to 40° C.
3. After mixing at this temperature for 90 minutes, the mixture is cooled to room temperature; when room temperature is reached mixing is stopped.
4. A sample is taken to verify the formation of the crystal liquid network.
5. In a second kettle, the oil phase ingredients are mixed with moderate agitation.
6. Once the formation of the crystal liquid in the main kettle is verified, the oil phase mixture is added.
7. Once the oil phase is completely added, an additional 3% of ion exchange resin may be added. Mixing is conducted slowly, at speed sufficient to maintain the ion exchange resin in suspension, continuing for 2 hours.
8. When the combination is uniform in appearance, the mixing is stopped.
9. The formation of the crystal liquid is again verified.
10. Any ion exchange resin present in the formulation is filtered out, and the formulation is filled into the chosen package.

### Example 2

This example illustrates the use of compositions of the invention in color correcting skin.

A group of 31 panelists was selected, and panelists were separated into groups depending upon their skin tone: 12 dark or olive complexions, 10 ruddy complexions, and 9 light complexions. On the day of testing, the panelists were told to report with no products on the face. Each group was treated with a color corrector appropriate to their skin type, dark or olive complexions receiving a gold color corrector, ruddy complexions receiving a red color corrector, and light complexions receiving an orange color corrector. The product was applied by spraying each side of the face with two sprays from a mist pump, after which the panelist lightly blended the mist into the skin. Evaluations were carried out before treatment (baseline) and immediately after product application. The effect of product application on skin tone was assessed and documents with close-up photography. Photos of the right and left side of the face were taken with a Nikon M3 digital camera. Panelists' heads are placed in a headrest to ensure reproducibility of positioning. The camera is positioned 2 feet from the panelist at an F stop of 32. Photos are evaluated by the investigator.

The results of the evaluation show that the gold color corrector improved the appearance of dark or olive colored skin by making the treated skin appear much brighter and less dull/gray after product application. Similarly, use of the orange color corrector on light skin caused the treated skin to appear brighter after application. Finally the red color corrector visibly reduced the amount of redness and brightened the skin on the treated panelists; however, in certain panelists, the corrector accentuated the appearance of broken capillaries.

The color correcting formulas used in the testing above are as follows(all amounts are in weight percent):

| Material | Gold | Red | Orange |
|---|---|---|---|
| Fire Crystals 615 (85% | 25.50 | 17.00 | 13.50 |
| water/15% silicone dioxide) | | | |
| Caprylyl | 1.00 | 1.00 | 1.06 |
| glycol/phenoxyethanol/hexylene | | | |
| glycol | | | |
| Butylene glycol | 5.10 | 5.10 | 5.40 |
| Purified water | 53.40 | 61.90 | 70.04 |
| Methyl perfluorobutyl ether | 6.00 | 6.00 | 4.00 |
| Cyclomethicone | 2.25 | 2.25 | 1.50 |
| Dimethicone, 200 cs | 2.25 | 2.25 | 1.50 |
| Cyclomethicone/polysilicone 11 | 4.50 | 4.50 | 3.00 |

Testing similar to that conducted above using oil-containing formulas was also conducted using different colored aqueous phases alone, each corrector tested on a smaller number of panelists, to determine which color corrector worked best on the panelist's individual type of skin tone. It was observed that on the two panelists, of different age groups, with dark skin, the gold color corrector produced the most beneficial effect, with the red corrector providing some benefit, while the orange was too orange on the skin. On the two panelists of different age groups having reddish skin, the red correct produced the best result, with the orange correct providing lesser benefits, and the gold corrector resulting in too strong a red color. The final panelist, a young woman with light skin, showed the most benefit with the orange corrector, the gold providing lesser benefits and the red resulting in an undesirable red color.

### Example 3

The following represent various multiphase formulations of the present invention:

A. Two Layers, Oil on Top:

| Material | Weight percent |
|---|---|
| Cyclomethicone | 19.75 |
| Bulgarian rose oil | 0.25 |
| Silicone dioxide | 2.40 |
| Purified water | 77.60 |

B. Two Layer, Oil on Bottom

| | |
|---|---|
| Methyl perfluorobutyl ether | 10.00 |
| Cyclomethicone | 10.00 |
| Silicone dioxide | 2.40 |
| Purified water | 77.60 |

C. Three Layers, Crystal Liquid on Bottom

| | |
|---|---|
| Apricot oil | 20.00 |
| Dimethicone, 350 cs | 30.00 |
| Silicone dioxide | 1.50 |
| Purified water | 48.50 |

D. Three Layers Crystal Liquid in the Center

| | |
|---|---|
| perfluoro-1,3-dimethylcyclohexane | 35.00 |
| Isododecane | 35.00 |
| Silicone dioxide | 0.90 |
| Purified water | 29.10 |

E. Three Layers, Crystal Liquid in the Center:

| | |
|---|---|
| Jojoba oil | 35.00 |
| Methyl perfluorobutyl ether | 35.00 |
| Silicone dioxide | 0.90 |
| Purified water | 29.10 |

### EXAMPLE 4

This example illustrates the enhanced delivery of an active utilizing a composition of the invention.

Experimental Design/Clinical Test Procedure:

Ten subjects free of any dermatological disorders and void of any marks, scratches, or bruises on their forearms are qualified for this study. The subjects were instructed not to use any products before reporting for testing. They are instructed to wash their arms with water 1-2 hours before reporting for testing. They remain at the testing center for 5 hours.

One lower arm is treated with approximately 2 mg/cm² of a composition of the invention and the other arm with a conventional emulsion composition, each containing Vitamin E. Twenty sequential tape strippings are collected from each arm immediately after product treatment and after 4 hours, under the conditions described below.

Sample Collection for Skin Penetration Via Tape Stripping:

Scotch tape strippings are collected, by placing a template over the area to be stripped and each tape is pressed on the skin within the outlined template area.

The tape is removed by gently pulling in a downward direction. The procedure is repeated twenty times at each sample collection interval. A new adjacent site is stripped at each sample collection interval. Every ten strippings are pooled and labeled according to subject name and time point. Upon collection the samples are placed in 2 milliliters of solvent and analyzed for the partitioning of the vitamin E into the stratum corneum.

Analytical Procedure:

The vitamin E acetate is extracted from the tape strips using 2 milliliters of methanol. The samples are run neat. The vitamin E acetate is quantified using an HPLC system specifically set up for vitamin E acetate. The method determines that there are no interferences between vitamin E acetate and that of sample excipients such as vehicle components, scotch tape, and typical biological components.

Results:

The data show that from the composition of the invention most of the vitamin E acetate (55%) is recovered in layers 1-10 and a small amount (8%) was recovered in layers 11-20. From the conventional emulsion, only 10% on the vitamin E acetate was recovered in each set of layers 1-10 and 11-20.

1. A topical system for application to the skin comprising an effective amount of a colloidal crystalline array in a hydrophilic phase, and at least one oil-containing phase

2. The system of claim 1 in which the array is composed of silica particles.

3. The system of claim 1 in which the oil phase comprises at least one silicon oil.

4. The system of claim 1 in which the oil phase also comprises a polyfluorinated solubilizer.

5. The system of claim 1 in which hydrophilic phase and the oil phase are present together in a single chamber of a dispensing package.

6. The system of claim 1 in which the hydrophilic phase and the oil phase visually appear as a single phase.

7. The system of claim 1 in which the hydrophilic phase and the oil phase are in physical contact and appear visually distinct from each other.

8. The system of claim 1 in which the hydrophilic phase and oil phase are contained in separate chambers of a single dispensing package.

9. The system of claim 1 in which they hydrophilic phase and the oil phase are contained in separate dispensing packages.

10. A topical system for application to the skin comprising an effective amount of a colloidal crystalline array comprising silica particles in a hydrophilic phase, and at least one oil-containing phase.

11. The system of claim 10 in which the silica particles have an average diameter of from about 50 to about 90 nanometers.

12. The system of claim 10 in which the silica particles have an average diameter of from about 60 to about 80 nanometers.

12. The system of claim 10 in which the particles are present in the hydrophilic phase in an amount of from about 0.5 to about 20% by weight of the hydrophilic phase.

13. The system of claim 10 which comprises at least 5% of an oil phase by weight of the total system.

14. The system of claim 13 which comprises at least one silicone oil.

15. The system of claim 14 which comprises a polyfluorinated solubilizer.

16. A topical system for application to the skin comprising an effective amount of a colloidal crystalline array comprising silica particles having an average diameter of from about 60 to about 80 nanometer in a hydrophilic phase, and at least one silicone oil-containing phase.

17. The system of claim 16 in which the oil phase contains a polyfluorinated solubilizer selected from the group consisting of perfluorocycloalkanes, hydrofluoroethers, perfluoromorpholines and perfluoroalkanes.

18. The system of claim 16 which also comprises at least one skin care active.

19. The system of claim 16 which also comprises a fragrance.

20. A method for modifying the appearance of color of a skin tone in an individual in need of such modification which comprises applying to the skin of the individual a composition comprising a colloidal crystalline array which is capable of filtering white light so as to permit a single color light to pass through, wherein the light passing through is a gold light, a red light or an orange light.

21. The method of claim 20 wherein the red light is achieved by a composition comprising in an aqueous phase from about 2.7% to about 3.9% by weight of silica particles having an average diameter of from about 60 to about 80 nanometers.

22. The method of claim 20 wherein the orange light is achieved by a composition comprising in an aqueous phase from about 0.75% to about 0.6% of silica particles having an average diameter of from about 60 to about 80 nanometers.

23. The method of claim 20 wherein the gold light is achieved by a composition comprising in an aqueous phase from about 4% to about 6% of silica particles having an average diameter of from about 60 to about 80 nanometers.

24. A method for delivering a skin care active to the skin which comprises applying to the skin the system of claim 1 in which the active is incorporated.

25. The method of claim 24 in which an active is incorporated into the hydrophilic phase.

26. The method of claim 24 in which an active is incorporated into the oil-containing phase.
